(19) United States
(12) Patent Application Publication
(10) Pub. No.: US 2021/0397978 A1
(43) Pub. Date: Dec. 23, 2021

(54) APPARATUS AND METHOD FOR PROCESSING DATA DISCOVERING NEW DRUG CANDIDATE SUBSTANCE

(71) Applicant: MEDIRITA, Seoul (KR)

(72) Inventors: Young Woo PAE, Seoul (KR); Seung-Hyun JIN, Seoul (KR)

(73) Assignee: MEDIRITA, Seoul (KR)

(21) Appl. No.: 17/288,905

(22) PCT Filed: Mar. 13, 2019
(86) PCT No.: PCT/KR2019/002918

§ 371 Date: Apr. 26, 2021

(30) Foreign Application Priority Data
Dec. 24, 2018 (KR) 10-2018-0168653
Mar. 13, 2019 (KR) 10-2019-0028788

(51) Int. Cl. G06N 5/02; G16C 20/40; G16C 20/70; G16C 20/50; G16C 20/90; G06K 9/62

(52) U.S. Cl. CPC G06N 5/022; G16C 20/40; G16C 20/70; G06K 9/6215; G16C 20/90; G06K 9/6267; G16C 20/50

(57) ABSTRACT

A method for processing data for discovering a new drug candidate substance by a data processing apparatus, includes receiving a predetermined search word, extracting at least one biological entity related to the predetermined search word from a big data database (DB), extracting a degree of mutual association between the predetermined search word and the at least one biological entity, generating a first knowledge network in which a plurality of nodes including the predetermined search word and the at least one biological entity are connected according to the degree of mutual association, computing a graph theory index of the first knowledge network, and generating a second knowledge network using some nodes of the plurality of nodes of which the graph theory index is equal to or greater than a threshold value.

### TECHNICAL FIELD

The present invention relates to an apparatus and method for processing data for discovering a new drug candidate substance, and more particularly, to an apparatus and method for processing data for generating a knowledge network from big data in order to discover the new drug candidate substance.

### BACKGROUND ART

It is known that it takes a total of 15 years and costs 2 to 3 trillion won on average to develop a new drug. Most of all, it is known that it takes about 6 years to discover the new drug candidate substance before preclinical trial.

In general, in order to discover the new drug candidate substance, which is a first stage in a pipeline for developing the new drug, a large number of specialized research personnel are going through a process of searching for a huge amount of information one by one and inferring association between major biological entities from this search.

Meanwhile, according to the Life Intelligence Consortium (2017) recently launched in Japan, when artificial intelligence technology is used to develop the new drug, it is predicted that the time required to develop a new drug can be reduced to about 40% and the cost can be reduced to about 50%.

However, a specific method for this has not been developed yet.

### DISCLOSURE OF THE INVENTION

### Technical Problem

A technical problem to be solved by the present invention is to provide an apparatus and method for processing data for discovering a new drug candidate substance.

Another technical problem to be solved by the present invention relates to an apparatus and method for generating a refined knowledge network from a big data DB.

### Technical Solution

A method for processing data for discovering a new drug candidate substance by a data processing apparatus according to an embodiment of the present invention includes receiving a predetermined search word, extracting at least one biological entity related to the predetermined search word from a big data database (DB), extracting a degree of mutual association between the predetermined search word and the at least one biological entity, generating a first knowledge network in which a plurality of nodes including the predetermined search word and the at least one biological entity are connected according to the degree of mutual association, computing a graph theory index of the first knowledge network, and generating a second knowledge network using some nodes of the plurality of nodes of which the graph theory index is equal to or greater than a threshold value.

The predetermined search word may include at least one of a gene name, a protein name, a metabolite name, a symptom name, a disease name, a compound name, and a drug name.

The biological entity may include at least one of genes, proteins, metabolites, symptoms, diseases, compounds, and drugs.

The biological entity and the first degree of mutual association may be extracted using at least one of a natural language processing algorithm and a deep neural network algorithm.

The big data DB may include at least one of a language-based DB for each type of biological entity and an image-based DB for each type of biological entity.

The graph theory index may include at least one of a shortest path between nodes, a clustering coefficient for each node, a centrality coefficient for each node, and a characteristic of a hub for each node for a plurality of nodes constituting the first knowledge network.

In the generating the second knowledge network, a standard score for each node may be computed using at least one of the shortest path between nodes, the clustering coefficient for each node, the centrality coefficient for each node, and the characteristic of the hub for each node for a plurality of nodes constituting the first knowledge network among the plurality of nodes, and a node having the standard score less than the threshold value may be deleted, and a connection associated with the deleted node may be deleted.

The standard score may be a value obtained by dividing a difference between an index value of a predetermined graph theory index for each node constituting the first knowledge network and an average index value of a predetermined graph theory index for the plurality of nodes constituting the first knowledge network by a standard error, and the threshold value may be 95% of significance.

An apparatus for processing data for discovering a new drug candidate substance according to an embodiment of the present invention includes a search word receiving unit that receives a predetermined search word, a data extracting unit that extracts at least one biological entity related to the predetermined search word from a big data database (DB), and extracts a degree of mutual association between the predetermined search word and the at least one biological entity, a data generating unit that generate a first knowledge network in which a plurality of nodes including the predetermined search word and the at least one biological entity are connected according to the degree of mutual association, a data processing unit that computes a graph theory index of the first knowledge network, a data refining unit that generates a second knowledge network using some nodes of the plurality of nodes of which the graph theory index is equal to or greater than a threshold value, and an output unit that exposes the second knowledge network.

A recording medium according to an embodiment of the present invention is a recording medium in which a computer-readable program is recorded in order to execute a data processing method which includes receiving a predetermined search word, extracting at least one biological entity related to the predetermined search word from a big data database (DB), extracting a degree of mutual association between the predetermined search word and the at least one biological entity, generating a first knowledge network in which a plurality of nodes including the predetermined search word and the at least one biological entity are connected according to the degree of mutual association, and computing a graph theory index of the first knowledge network, and generating a second knowledge network using some nodes of the plurality of nodes of which the graph theory index is equal to or greater than a threshold value.

### Advantageous Effects

According to the embodiment of the present invention, refined information on biological entities related to a predetermined search word and a degree of mutual association between the biological entities can be extracted within a short time without searching for a huge amount of information one by one in order to discover new drug candidate substance. Accordingly, it is possible to significantly reduce the cost and period required to discover a new drug candidate substance or a target of new drug candidate substance.

### BRIEF DESCRIPTION OF THE DRAWINGS

FIG. 1 is a block diagram of an apparatus for processing data for discovering a new drug candidate substance according to an embodiment of the present invention;

FIG. 2 is a flowchart of a method for processing data for discovering a new drug candidate substance of the apparatus for processing data according to an embodiment of the present invention;

FIG. 3 illustrates an example in which a predetermined search word is input;

FIG. 4 illustrates a part of an example of a matrix representing biological entities extracted in step S110 and degrees of mutual associations between the biological entities extracted in step S120;

FIG. 5 illustrates a part of an example of categories of the degrees of mutual association for extracting the matrix of FIG. 4;

FIG. 6 illustrates an example of a first knowledge network generated according to an embodiment of the present invention;

FIG. 7 illustrates an example of classifying types of hubs according to a participation coefficient (PC); and

FIG. 8 illustrates an example of a second knowledge network generated by using “epilepsy syndrome” as a search word according to an embodiment of the present invention.

### MODE FOR CARRYING OUT THE INVENTION

The present invention can be modified in various ways and may include various embodiments, and thus specific embodiments of the present invention will be described by exemplifying them in the drawings. However, this is not intended to limit the present invention to the specific embodiments and should be understood to cover all changes, equivalents, and substitutes included within the spirit and technical scope of the present invention.

Terms including ordinal numbers such as second and first may be used to describe various components, but the components are not limited by the terms. The above terms are used only for the purpose of distinguishing one component from another component. For example, without departing from the scope of right of the present invention, a second component may be named as a first component, and similarly, the first component may be named as the second component. The term of and/or includes a combination of a plurality of related listed items or any of the plurality of related listed items.

When a certain component is referred to as being “coupled” or “connected” to another component, it should be understood that the component may be directly coupled or connected to the other component, but other components may exist in the middle. On the other hand, when a certain component is referred to as being “directly coupled” or “directly connected” to another component, it should be understood that there is no other component in the middle.

The terms used in the present application are only used to describe specific embodiments, and are not intended to limit the present invention. A singular expression includes a plural expression, unless it is explicitly meant differently in the context. In the present application, it is to be understood that terms such as “include” or “have” are intended to designate the existence of features, numbers, steps, actions, components, parts, or combinations thereof described in the specification and do not preclude the possibility of the presence or addition of one or more other features or numbers, steps, actions, components, parts, or combinations thereof.

Unless otherwise defined, all terms used herein including technical or scientific terms have the same meaning as generally understood by a person with ordinary skill in the art to which the present invention pertains. Terms such as those defined in a generally used dictionary, should be interpreted as having a meaning consistent with the meaning in the context of the related technology, and are not to be interpreted as an ideal or excessively formal meaning unless explicitly defined in the present application.

Hereinafter, exemplary embodiments will be described in detail with reference to the accompanying drawings, but identical or corresponding components are denoted by the same reference numerals regardless of reference symbols, and redundant descriptions thereof will be omitted.

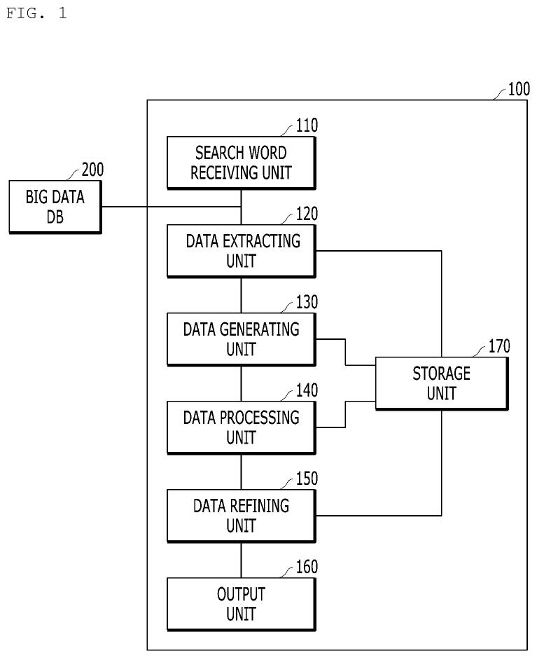

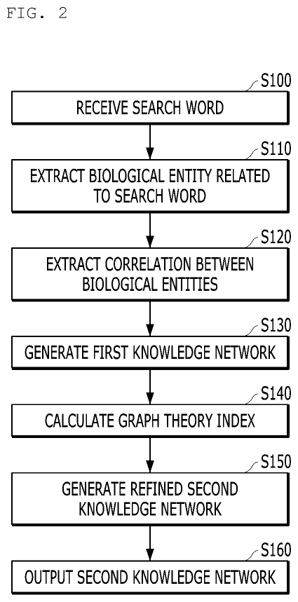

FIG. 1 is a block diagram of an apparatus for processing data for discovering a new drug candidate substance according to an embodiment of the present invention, and FIG. 2 is a flowchart of a method for processing data for discovering a new drug candidate substance of the apparatus for processing data according to an embodiment of the present invention.

Referring to FIG. 1, an apparatus for processing data 100 for discovering a new drug candidate substance includes a search word receiving unit 110, a data extracting unit 120, a data generating unit 130, a data processing unit 140, and a data refining unit 150, an output unit 160, and a storing unit 170.

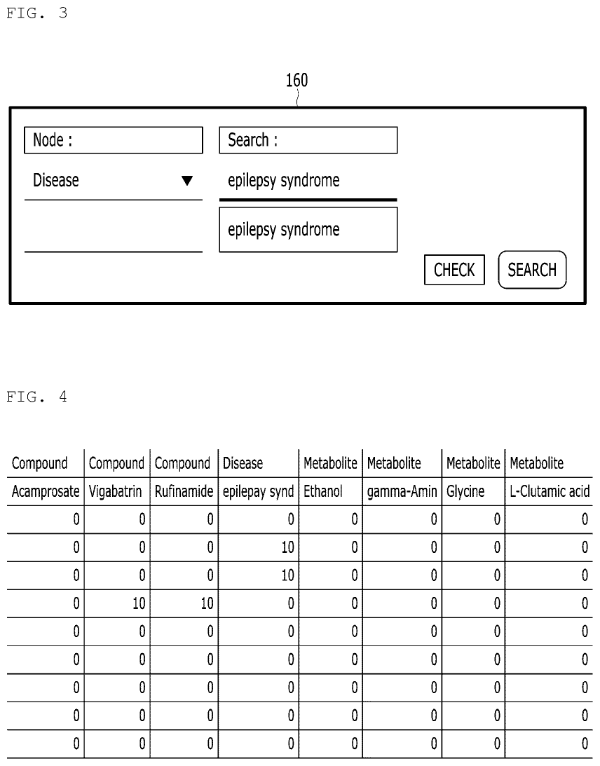

Referring to FIGS. 1 to 2, the search word receiving unit 110 receives a predetermined search word (S100). The predetermined search word may be a search word that a user wants to search for information, and may be input through a user interface, and may include at least one of a gene name, a protein name, a metabolite name, a symptom name, a disease name, a compound name, and a drug name. FIG. 3 illustrates an example in which the predetermined search word is input. Referring to FIG. 3, a screen for inputting the predetermined search word may be exposed through the output unit 160, and the predetermined search word may be input through the user interface. FIG. 3 illustrates an example in which a disease name is selected as a category and epilepsy syndrome is input as the predetermined search word.

Next, the data extracting unit 120 extracts at least one biological entity related to the predetermined search word received in step S100 (S110), and extracts a degree of mutual association between the predetermined search word and the extracted biological entity (S120). Here, the biological entity may include at least one of genes, proteins, metabolites, symptoms, diseases, compounds, and drugs, and a level to which the predetermined search word belongs may be the same as or different from a level to which the biological entity belongs. For example, as illustrated in FIG. 3, when the predetermined search word is epilepsy syndrome, which is a disease name, the biological entities extracted in step S110 may include at least one of genes associated with epilepsy syndrome, proteins associated with epilepsy syndrome, metabolites associated with epilepsy syndrome, symptoms associated with epilepsy syndrome, diseases associated with epilepsy syndrome, compounds associated with epilepsy syndrome, and drugs associated with epilepsy syndrome. In addition, the biological entities extracted in step S110 may include a plurality of biological entities for each level. For example, as illustrated in FIG. 3, when the predetermined search word is epilepsy syndrome, which is a disease name, the biological entities extracted in step S110 may include at least one of a plurality of genes associated with epilepsy syndrome, a plurality of proteins associated with epilepsy syndrome, a plurality of metabolites associated with epilepsy syndrome, a plurality of symptoms associated with epilepsy syndrome, a plurality of diseases associated with epilepsy syndrome, a plurality of compounds associated with epilepsy syndrome, and a plurality of drugs associated with epilepsy syndrome.

To this end, the data extracting unit 120 may use a big data DB 200. The big data DB 200 may be a DB outside the apparatus for processing data 100 according to the embodiment of the present invention, and may be a global public DB that anyone can access or can be accessed by a person who has been authenticated under predetermined conditions. The big data DB 200, may be store information about biological entities and a degree of mutual association between biological entities in advance. For example, the big data DB 200 may include a DB for each type of biological entity and a DB for the degree of mutual association between biological entities. The DB for each type of biological entity may include a gene DB, a protein DB, a metabolite DB, a symptom DB, a disease DB, a compound DB, and a drug DB. These DBs may be managed and operated by being integrated into one big data DB, or managed and operated by being distributed. In this specification, the big data DB 200 may be used interchangeably with an omics DB.

In this case, in order for the data extracting unit 120 to extract at least one biological entity related to a predetermined search word and a degree of mutual association between biological entities, the data extracting unit 120 may be based on artificial intelligence technology including machine learning and use a natural language processing algorithm. Here, natural language processing refers to various technologies that mechanically analyze language phenomena spoken by humans to make them into a form that can be understood by a computer, and express the form that can be understood by the computer in a language that can be understood by humans. To this end, the big data DB 200 may be a language-based DB for each type of biological entity, and may include information reflecting machine learned results and feedback results.

Alternatively, in order for the data extracting unit 120 to extract at least one biological entity related to the predetermined search word and the degree of mutual association between biological entities, the data extracting unit 120 may be based on artificial intelligence technology including machine learning, and may use a deep neural network algorithm. Here, the deep neural network is an artificial neural network composed of several hidden layers between an input layer and an output layer, and refers to various technologies used for classification, prediction, image recognition, character recognition, etc. To this end, the big data DB 200 may be an image-based DB for each type of biological entity, and may include information reflecting machine learned results and feedback results.

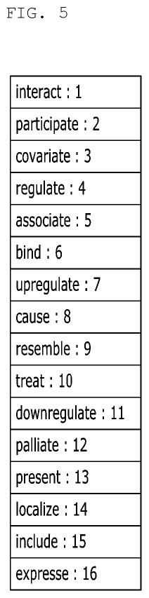

FIG. 4 illustrates a part of an example of a matrix representing biological entities extracted in steps S110 and S120 and degrees of mutual associations between the biological entities and FIG. 5 illustrates a part of an example of categories of the degrees of mutual association for extracting the matrix of FIG. 4.

Referring to FIGS. 4 to 5, the categories of the degrees of mutual associations between biological entities may include “interact”, “participate”, “covariate”, “regulate”, “associate”, “bind”, “upregulate”, “cause”, “resemble”, “treat”, “downregulates”, “palliate”, “present”, “localize”, “include”, “express”, etc., and an identification number may be randomly assigned for each category. The identification number for each category may be set by the user or may be set automatically.

For example, when bupropion, which is a drug name, is received as the predetermined search word in step S100, the data extracting unit 120 may extract “acamprosate”, “vigabatrin”, and “rufinamide” as a compound related to bupropion, may extract “epilepsy syndrome” as a disease, and extract “ethanol”, “gamma-amine”, “glycine”, “L-glutamic acid”, etc. as metabolites, and may generate a matrix in which a category of a degree of mutual association between a predetermined search word and a biological entity or a category of a degree of mutual association between biological entities is represented by an identification number. In the matrix of FIG. 4, the first column represents the categories of biological entities, the second column represents the biological entities extracted for each category, and the numbers in the lower columns represent the categories of the degrees of mutual association. The form of such a matrix is exemplary, and is not limited thereto, and may be modified in various forms.

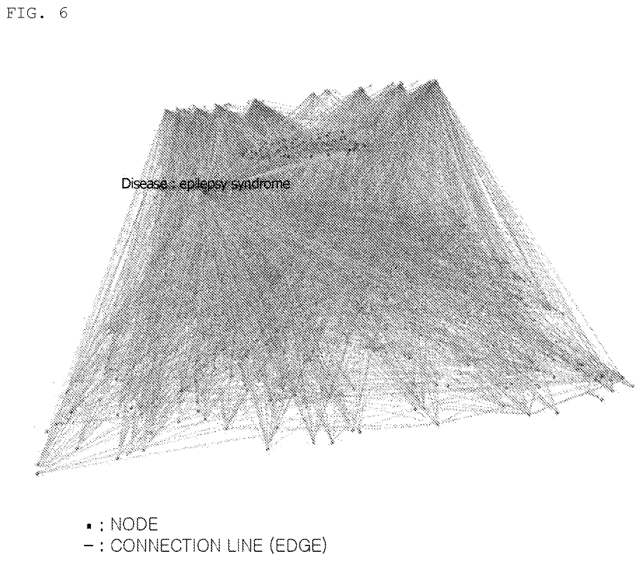

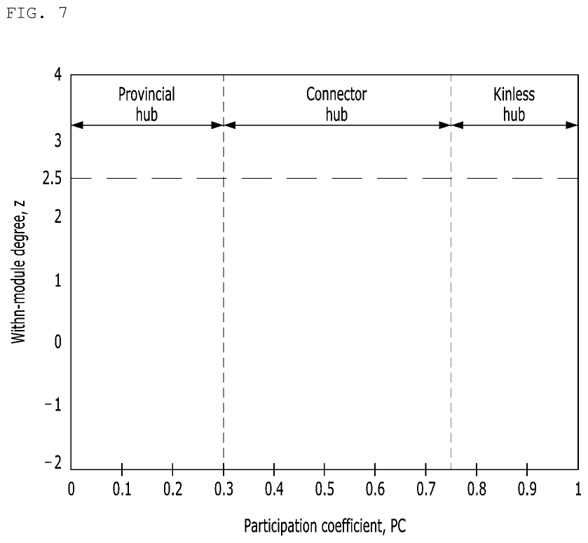

Next, the data generating unit 130 generates a first knowledge network using the results extracted in steps S110 and S120 (S130). FIG. 6 illustrates an example of a first knowledge network generated according to an embodiment of the present invention. Here, the first knowledge network may have a form in which the predetermined search word received in step S100 and each of at least one biological entity extracted in step S110 are used as nodes, and a plurality of nodes are connected using connection lines according to the degrees of mutual associations between the predetermined search word and the biological entities extracted in step S120 or the degrees of mutual associations between the biological entities. The connection lines may connect nodes within the same omics level or may connect nodes within different omics levels. Paths from node A, which is one of nodes in the first knowledge network, to node B, which is the other one thereof, may vary, and all possible paths may be connected by the connection lines. Here, the knowledge network is a network composed of the degrees of mutual associations between the biological entities, and may be used interchangeably with a biological network.

Next, the data processing unit 140 computes graph theory indexes of the first knowledge network generated in step S130 (S140). Here, the graph theory indexes may include at least one of a shortest path between nodes, a clustering coefficient for each node, a centrality coefficient for each node, and a hub characteristic for each node for a plurality of nodes constituting the first knowledge network.

The shortest path between nodes may mean the shortest path among a large number of paths from node A to node B in the first knowledge network. Hereinafter, a method of calculating the shortest path between node A, which is one of the biological entities, and node B, which is the other of the biological entities, will be described. There are various paths from node A to node B, and node A and node B may be directly connected, or at least one intermediate node may exist on each path between node A and node B.

The shortest path between node A and node B can be obtained by using the number of intermediate nodes for each path. For example, among various paths between node A and node B, a path with a smaller number of intermediate nodes may be determined to be a shorter path.

Or, the shortest path between node A and node B may be obtained using the number of intermediate nodes for each path, but may reflect a type of mutual association for each connection line. That is, weights may be set differently for each category of mutual association, and the weights may also be applied to mutual association that exists for each path. The types of mutual associations are as illustrated in FIG. 5 and may have different weight values for each type of mutual association.

Equation 1 is an example of an equation for calculating the shortest path between nodes.

d_{i,j}^{W}=Σ_{w}_{ₛₜ}_{∈g}_{_{i→j}}_{w}f(wₛₜ) [Equation 1]

Here, wₛₜ is a mutual association index between two nodes s and t, f is a weight transformation function, and g_{i→j}^{w} is the shortest path between two nodes i and j. A value of Equation 1 is obtained for each path, and a path having the lowest value or the highest value may be selected as the shortest path.

Next, the clustering coefficient for each node may be computed by Equation 2 and Equation 3. Here, the clustering coefficient may be referred to as a grouping coefficient, and may mean a probability that a specific node and neighboring nodes are connected to each other or a connection density between the specific node and neighboring nodes.

tᵢ^{w}=½Σ_{j,h∈N}wᵢⱼwᵢₕwⱼₕ [Equation 2]

Here, tᵢ^{w} means the number of triangles in a graph created around each node i of the knowledge network, N is the total set of nodes in the knowledge network, wᵢⱼ is a mutual association index between two nodes i and j, wₜₕ is a mutual association index between nodes i and h, and wᵢₕ is a mutual association index between two nodes j and h.

$${C}^{w} = \frac{1}{n} {\sum }_{i ∈ N}^{ } \frac{2 {t}_{i}^{w}}{ki ( ki - 1 )} \qquad [ Equation 3 ]$$

Here, C^{w} means the clustering coefficient, tᵢ^{w} is the number of triangles in the graph created around each node i of the knowledge network, and kᵢ means a degree of node i, that is, a value of the degree of connectivity of node i in the knowledge network.

Next, the centrality index for each node is an index of whether a specific node has the function of a hub, and may be expressed by a D_{nodal} (nodal degree) value, a betweenness centrality (BC) value, a E_{nodal} (nodal efficiency) value, etc. Here, the D_{nodal} value is a value of the degree of connectivity of each node in the knowledge network, that is, an index indicating how strong or weak node i has connectivity in the knowledge network, the E_{nodal} value is a value of a degree of efficiency of node i in the knowledge network, that is, a value expressed as the reciprocal of the shortest path of Equation 1, and is a value with higher efficiency as the path is shorter, and the BC value is an index indicating the number of times that node i becomes a shortcut in the path between nodes in the knowledge network.

First, the D_{nodal} value may be computed by Equation 4.

D_{nodal}(i)=Σ_{j∈N}wᵢⱼ [Equation 4]

Here, wᵢⱼ is a mutual association index between two nodes i and j, and N is a total set of nodes in the knowledge network.

In addition, the E_{nodal} value may be computed by Equation 5.

$${E}_{nodal} ( i ) = {\sum }_{j ∈ N , j \neq i}^{ } \frac{1}{{d}_{i , j}^{w}} \qquad [ Equation 5 ]$$

Here, N is a total set of nodes of the knowledge network, and d_{i,j}^{w} is a value indicating the shortest path computed in Equation 1.

Next, Betweenness centrality (BC) may be computed by Equation 6.

$$BC ( i ) = {\sum }_{{h , j ∈ N}_{h \neq j , h \neq i , j \neq i}}^{ } \frac{{g}_{hj} ( i )}{{g}_{hj}} \qquad [ Equation 6 ]$$

Here, gₕⱼ means the shortest distance between nodes h and j, and gₕⱼ(i) means the shortest distance between h and j passing through node i.

Next, when it is determined that a predetermined node has a function of a hub, characteristics of the hub are classified. In this case, the characteristics of the hub may be classified into a kinless hub, a connector hub, a provincial hub, etc. Here, the kinless hub means a hub with the most influential hub, that is, a hub connected to nodes in many modules, the connector hub means a hub that connects modules in the knowledge network, and the provincial hub means a hub that has a high influence mainly within the module. Here, the module may be a structural configuration group obtained by subdividing the entire knowledge network.

To this end, a modularity in the knowledge network can be computed as in Equation 7. The modularity means the number of module types in the entire knowledge network.

$${Q}^{W} = \frac{1}{{l}^{W}} {\sum }_{i , j ∈ N}^{ } [ {w}_{ij} - \frac{{k}_{i}^{W} {k}_{j}^{W}}{{l}^{W}} ] {δ}_{mi , mj} \qquad [ Equation 7 ]$$

Here, kᵢⁿΣ_{j∈N}wᵢⱼ means the sum of weights at node i, and l^{w}=Σ_{i,j∈N}wᵢⱼ means the sum of weights. δ_{mi,mj} is the kronecker delta, 1 for mi=mj, and 0 for the rest. Next, the participation coefficient (PC) of the knowledge network module may be computed as in Equation 8.

$${PC}_{i} = 1 - {\sum }_{m ∈ M}^{ } {( \frac{{k}_{i}^{w} ( m )}{{k}_{i}^{w}} )}^{2} \qquad [ Equation 8 ]$$

Here, M means a set of modules, kᵢ^{w}(mᵢ) means the number of connections between node i and all other nodes in module m, and module m means a structural group obtained by subdividing the entire knowledge network.

In addition, a z score (within-module degree) of the knowledge network module may be computed as in Equation 9.

$${z}_{i}^{W} = \frac{{k}_{i}^{W} ( mi ) - {\overline{k}}^{W} ( mi )}{{σ}_{k}^{W} ( mi )} \qquad [ Equation 9 ]$$

Here, mᵢ means node i in module m, kᵢ^{w}(mᵢ) means the degree of connectivity in module m of node i, and k̅^{w}(mᵢ) and σₖ^{W}(mᵢ) refer to the mean and standard deviation of the degree distribution of connectivity within module m, respectively.

Through the computation of the indexes in Equation 9 above, it is possible to distinguish whether each node is a hub or not within the module. For example, as follows, when the Z score of the knowledge network module is 2.5 or higher, it may be determined as a hub.

1. within-module z-score≥2.5: hub

2. within-module z-score<2.5: not hub

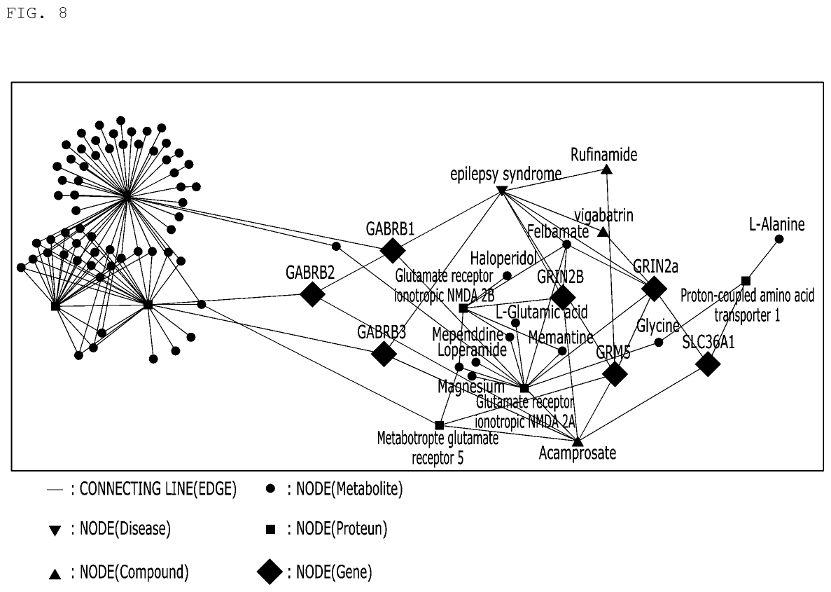

In addition, when it is determined that the node is a hub in the module, a type of the hub can be classified as follows through the computation of the indexes in Equation 8, and FIG. 7 illustrates an example of classifying the types of the hub according to PCs.

1. Provincial hub: PC≤0.30

2. Connector hub: 0.3<PC≤0.75

3. Kinless hub: PC>0.75

In this way, when the data processing unit 140 computes the graph theory index in step S140, the data refining unit 150 generates a second knowledge network refined from the first knowledge network using the graph theory index (S150). Here, the second knowledge network is a network that is more simplified than the first knowledge network, and may be composed of only some nodes having high correlation in terms of the graph theory among a plurality of nodes constituting the first knowledge network.

In this case, the nodes constituting the second knowledge network may be some nodes of which at least a part of an index value for the shortest path between nodes, an index value for the clustering coefficient for each node, and an index value for the centrality coefficient for each node is equal to or greater than a threshold value among the graph theory indexes computed in step S140, among the plurality of nodes constituting the first knowledge network. That is, the second knowledge network may be generated in such a way of deleting the nodes of which at least a part of the index value for the shortest path between nodes, the index value for the clustering coefficient for each node, and the index value for the centrality coefficient for each node is less than the threshold value among the plurality of nodes constituting the first knowledge network and deleting the connections associated with the deleted nodes.

Here, the graph theory index compared to the threshold value may be the index value for the shortest path between nodes, the index value for the clustering coefficient for each node, and the index value for the centrality coefficient for each node. Alternatively, the graph theory index compared to the threshold value may be a value calculated by integrating at least two of the index value for the shortest path between nodes, the index value for the clustering coefficient for each node, and the index value for the centrality coefficient for each node.

In this case, at least one of the index value for the shortest path between nodes, the index value for the clustering coefficient for each node, and the index value for the centrality coefficient for each node may be computed as a standard score for each node, and the computed standard score may be compared with the threshold value.

Here, the standard score may be the z score, and the threshold value may mean 95% of significance.

The Z score can be computed as in Equation 10.

$$Z = \frac{X - mean ( x )}{SE ( x )} \qquad [ Equation 10 ]$$

Here, z is the z score, X is an index value of a predetermined graph theory index for a specific node in the first knowledge network, mean(x) is an average index value of predetermined graph theory indexes for a plurality of nodes in the first knowledge network, and SE(x) is a standard error of the index value of the predetermined graph theory index in the first knowledge network. Here, it can be expressed as SE=σ/\sqrt{n}, where σ is the standard deviation, and n is the number of nodes constituting the first knowledge network.

That is, the z score may be a value obtained by dividing the difference between the index value of the predetermined graph theory index for each node constituting the first knowledge network and the average index value of the predetermined graph theory index for the plurality of nodes constituting the first knowledge network by the standard error.

In this case, the z score may be computed through a permutation test. The permutation test may be performed by randomly mixing all connection lines constituting the first knowledge network and then computing the z score for each node. In this case, the number of times of random mixing of the connection lines may be 1000 times or more.

Or, the nodes constituting the second knowledge network may be some nodes extracted by using the index value for the hub characteristic for each node among the graph theory indexes computed in step S140 from among the plurality of nodes constituting the first knowledge network. That is, the node constituting the second knowledge network may be a node determined to be a hub within the module through the computation of the index of Equation 9, preferably a node classified as one of the kinless hub, the connector hub, and the provincial hub, more preferably a node classified as one of the kinless hub and the connector hub, and more preferably, a node classified as the kinless hub.

Next, the output unit 160 outputs the second knowledge network generated in step S150 (S160). The output unit 160 may be, for example, a display. FIG. 8 illustrates an example of the second knowledge network generated by using “epilepsy syndrome” as a search word according to an embodiment of the present invention. Referring to FIG. 8, it can be seen that the second knowledge network that is significantly simplified and refined compared to the first knowledge network of FIG. 6. In addition, referring to FIG. 8, it can be seen that biological entities within different omics levels associated with “epilepsy syndrome” and the mutual association between the biological entities can be intuitively obtained.

As described above, according to an embodiment of the present invention, it is possible to obtain the second knowledge network composed of only nodes refined related to a predetermined search word, and accordingly, a new drug candidate substance or a target of the new drug candidate substance can be easily determined.

Meanwhile, the apparatus for processing data 100 according to an embodiment of the present invention may include the data storing unit 170. The data storing unit 170 may be connected to the data extracting unit 120, the data generating unit 130, the data processing unit 140, and the data purification unit 150, and may store results calculated from the data extracting unit 120, the data generating unit 130, the data processing unit 140, and the data refining unit 150. The data storing unit 170 may be connected to an external learning server in a wired or wirelessly manner, and may transmit stored data to the external learning server.

The term ‘˜ unit’ used in this embodiment means software or hardware components such as field-programmable gate array (FPGA) or ASIC, and ‘˜ unit’ performs certain roles. However, the ‘˜ unit’ is not limited to software or hardware. The ‘˜ unit’ may be configured to be located in an addressable storage medium, or may be configured to reproduce one or more processors. Accordingly, as an example, the ‘˜ unit’ includes components such as software components, object-oriented software components, class components, and task components, processes, functions, attributes, procedures, subroutines, segments of program code, drivers, firmware, microcode, circuits, data, databases, data structures, tables, arrays, and variables. Components and functions provided in the ‘˜ units’ may be combined into a smaller number of components and ‘˜ units’, or may be further separated into additional components and ‘˜ units’. In addition, components and ‘˜ units’ may be implemented to play one or more CPUs in a device or a security multimedia card.

In the description above, although description has been made with reference to a preferred embodiment of the present invention, those skilled in the art will appreciate that various modifications and changes may be made to the present invention within a range not departing from the spirit and scope of the present invention set forth in the following claims.

1. A method for processing data for discovering a new drug candidate substance by a data processing apparatus, the method comprising:
receiving a predetermined search word;
extracting at least one biological entity related to the predetermined search word from a big data database (DB);
extracting a degree of mutual association between the predetermined search word and the at least one biological entity;
generating a first knowledge network in which a plurality of nodes including the predetermined search word and the at least one biological entity are connected according to the degree of mutual association;
computing a graph theory index of the first knowledge network; and
generating a second knowledge network using some nodes of the plurality of nodes of which the graph theory index is equal to or greater than a threshold value.

2. The method of claim 1,
wherein the predetermined search word includes at least one of a gene name, a protein name, a metabolite name, a symptom name, a disease name, a compound name, and a drug name.

3. The method of claim 1,
wherein the biological entity includes at least one of genes, proteins, metabolites, symptoms, diseases, compounds, and drugs.

4. The method of claim 1,
wherein the biological entity and the first degree of mutual association are extracted using at least one of a natural language processing algorithm and a deep neural network algorithm.

5. The method of claim 1,
wherein the big data DB includes at least one of a language-based DB for each type of biological entity and an image-based DB for each type of biological entity.

6. The method of claim 1,
wherein the graph theory index includes at least one of a shortest path between nodes, a clustering coefficient for each node, a centrality coefficient for each node, and a characteristic of a hub for each node for a plurality of nodes constituting the first knowledge network.

7. The method of claim 6,
wherein, in the generating the second knowledge network,
a standard score for each node is computed using at least one of the shortest path between nodes, the clustering coefficient for each node, and the centrality coefficient for each node for a plurality of nodes constituting the first knowledge network among the plurality of nodes, and
a node having the standard score less than the threshold value is deleted, and a connection associated with the deleted node is be deleted.

8. The method of claim 7,
wherein the standard score is a value obtained by dividing a difference between an index value of a predetermined graph theory index for each node constituting the first knowledge network and an average index value of a predetermined graph theory index for the plurality of nodes constituting the first knowledge network by a standard error, and the threshold value is 95% of significance.

9. An apparatus for processing data for discovering a new drug candidate substance, the apparatus comprising:
a search word receiving unit that receives a predetermined search word;
a data extracting unit that extracts at least one biological entity related to the predetermined search word from a big data database (DB), and extracts a degree of mutual association between the predetermined search word and the at least one biological entity;
a data generating unit that generate a first knowledge network in which a plurality of nodes including the predetermined search word and the at least one biological entity are connected according to the degree of mutual association;
a data processing unit that computes a graph theory index of the first knowledge network;
a data refining unit that generates a second knowledge network using some nodes of the plurality of nodes of which the graph theory index is equal to or greater than a threshold value; and
an output unit that exposes the second knowledge network.

10. The apparatus of claim 9,
wherein the predetermined search word includes at least one of a gene name, a protein name, a metabolite name, a symptom name, a disease name, a compound name, and a drug name.

11. The apparatus of claim 9,
wherein the biological entity includes at least one of genes, proteins, metabolites, symptoms, diseases, compounds, and drugs.

12. The apparatus of claim 9,
wherein the data extracting unit extracts the biological entity and the first degree of mutual association using at least one of a natural language processing algorithm and a deep neural network algorithm.

13. The apparatus of claim 9,
wherein the big data DB includes at least one of a language-based DB for each type of biological entity and an image-based DB for each type of biological entity.

14. The apparatus of claim 9,
wherein the graph theory index includes at least one of a shortest path between nodes, a clustering coefficient for each node, a centrality coefficient for each node, and a characteristic of a hub for each node for a plurality of nodes constituting the first knowledge network.

15. The apparatus of claim 14,
wherein the data refining unit computes a standard score for each node using at least one of the shortest path between nodes, the clustering coefficient for each node, and the centrality coefficient for each node for a plurality of nodes constituting the first knowledge network among the plurality of nodes, and
a node having the standard score less than the threshold value is deleted, and a connection associated with the deleted node is be deleted.

16. The apparatus of claim 14,
wherein the standard score is a value obtained by dividing a difference between an index value of a predetermined graph theory index for each node constituting the first knowledge network and an average index value of a predetermined graph theory index for the plurality of nodes constituting the first knowledge network by a standard error, and the threshold value is 95% of significance.

17. A recording medium in which a computer-readable program is recorded in order to execute a data processing method which includes:
receiving a predetermined search word;
extracting at least one biological entity related to the predetermined search word from a big data database (DB);
extracting a degree of mutual association between the predetermined search word and the at least one biological entity;
generating a first knowledge network in which a plurality of nodes including the predetermined search word and the at least one biological entity are connected according to the degree of mutual association;
computing a graph theory index of the first knowledge network; and
generating a second knowledge network using some nodes of the plurality of nodes of which the graph theory index is equal to or greater than a threshold value.
